(19) United States
(12) Patent Application Publication
(10) Pub. No.: US 2012/0211573 A1
(43) Pub. Date: Aug. 23, 2012

(54) Fragrance Dispenser For Use With Decorative Emanating Surface Botanical Products

(75) Inventors: Gerald Allison, East Windsor, NJ (US); Cheriyan B. Thomas, Farmington, CT (US)

(73) Assignee: The Yankee Candle Way, So. Deerfield, MA (US)

(21) Appl. No.: 13/369,784

(22) Filed: Feb. 9, 2012

Related U.S. Application Data
(60) Provisional application No. 61/443,890, filed on Feb. 17, 2011.

(51) Int. Cl. A61L 9/04

(52) U.S. Cl. 239/6; 239/44

(57) ABSTRACT

The invention relates to a fragrance dispenser for botanical products and methods of use thereof. The fragrance dispenser includes a holding member having a surface for holding a botanical product such as potpourri; a support member for supporting the holding member and for receiving a supply of liquid fragrance; and a plurality of wicking members extending from the supply of liquid fragrance to and upon the holding member surface for transferring the liquid fragrance from the supply to the surface for diffusing the fragrance therefrom.

### CROSS REFERENCE TO RELATED APPLICATION

This application claims priority to U.S. Provisional Application No. 61/443,890, filed on Feb. 17, 2011, which is incorporated herein by reference.

### BACKGROUND

The present invention relates to a fragrance dispenser that includes a botanical system with wicking devices attached to a continuous fragrance reservoir. This system maintains the air freshening quality of the product throughout the life of the fragrance reservoir. The traditional means of fragrance botanical potpourri is to spray the botanical with fragrance as the product rolls around in a tumbler. This method leads to poor fragrance absorption which in turn leads to a short shelf-life of the fragranced product with odor perceptible fragrances lasting approximately 2 weeks. This poor fragrance delivery ultimately leads to poor consumer acceptance and reception of the product.

Improvements in maintaining fragrance strength in potpourri botanicals, have been attempted. U.S. Pat. No. 6,249,645 discloses a scent device that is adapted for use in a vehicle and that includes a bowl having multiple compartments therein and a heater element for heating and dispensing a fragrance. The container can hold various potpourri scents therein as well as to allow the user to make a desired scent such as be brewing flowers in water in the container.

US patent application 2007/0187524 discloses a device to scent an environment comprising a scent and a structural component. The structural component is typically made of absorbent material in a rod-like shape having a length to average cross-sectional diameter ratio of at least about 4:1. The absorbent rod has a specific gravity of greater than about 0.95 in order to provide a slow release of scent. The scent stick can be colored to blend into an environmental setting to make the presence of the scent stick unobtrusive, and is used in live or artificial floral arrangements to boost the odor of the flowers.

Reed sticks in general are known and various improvements have been made upon them. US patent application 2009/0039174 discloses reeds that include a buoyant member so that they can float on a supply of fragrance to help dispense the fragrance.

Also, a wide variety of containers have been used to dispense aromas. These include multi-compartment containers such as those disclosed in US patent application 2006/0018786 and those having a central burn control aperture such as in U.S. Pat. No. 7,337,772.

While the foregoing devices and methods are useful, there still remains a need for enhancing the fragrance releasing properties of potpourri and the present invention now provides new devices and methods to meet that need.

### SUMMARY OF THE INVENTION

The invention relates to a fragrance dispenser comprising a holding member having a surface for holding a product, decorative elements, or an ornament or emanating surface that absorbs and releases fragrance; a support member for supporting the holding member and for receiving a supply of liquid fragrance; and a plurality of wicking members extending from the supply of liquid fragrance to and upon the holding member surface for transferring the liquid fragrance from the supply to the surface for diffusing the fragrance therefrom. The decorative elements, ornament(s), or emanating surface(s) or a product such as botanicals e.g., potpourri, are preferably provided on the holding member.

The supply of liquid fragrance is received in a fragrance chamber provided in the support member, with the holding member advantageously being shaped to retain the product, decorative elements, or an ornament or emanating surface therein or thereon with the support member being bowl to support the holding member. Preferably, the holding member and support member are bowl shaped. Alternatively, the holding member may be a dish or platter. The holding member preferably is designed to have a hole therein through which the wicking members pass to reach the supply of liquid fragrance. For either embodiment, the supply of liquid fragrance may be provided in a bottle with the fragrance chamber mounted in the support member beneath a hole in the holding member. Preferably, the fragrance chamber is mounted centrally in the support member beneath a centrally located hole in the holding member.

The holding member preferably has a generally circular perimeter with the plurality of wicking members comprising reed or other wicking members having first ends configured to extend into the supply of liquid fragrance and having second ends configured conform to the holding member surface and extend sufficiently towards the perimeter of the holding member. The wicking material is extended to provide greater surface area for fragrance diffusion. Preferably, the wicking members extend at least halfway towards the holding member perimeter. The second ends of the reed members extend to near the perimeter of the holding member and then in a different direction to extend over additional portions of the holding member surface for greater diffusion of the fragrance therefrom.

Generally, between 2 and 50 wicking members are provided, each having a diameter of about 0.05 to 0.5 inches and have a length sufficient to diffuse the desired amount of liquid fragrance. The wicking material can also be flat or in sheet form. These wicking members may be made of paper, a fabric or a polymer material capable of absorbing and releasing the fragrance and the holding member and support member are each made of plastic, ceramic, metal or other materials of combinations thereof such that the inner surface of the holding member does not absorb the liquid fragrance.

The invention also relates to a method for enhancing product fragrance diffusion, which comprises providing a fragrance of the type disclosed herein; adding a liquid fragrance to the support member; and arranging the wicking members to contact the supply of liquid fragrance to transfer the liquid fragrance from the supply to the surface of the holding member for diffusing the fragrance therefrom. The emanating surface can be or include botanicals such as potpourri or ornaments such as cellulose acetate pad, decorative paper, any polymer that will adsorb and release fragrance. This holding member can be a scented holding plate for any object. For example, the object can be a scented Christmas tree, a diffuser fruit basket, a scented garbage disposal holder for kitchen and bathroom, etc. Preferably, the method further comprises arranging a supply of potpourri as a botanical product upon the holding member so that it appears that the potpourri is emanating the fragrance. The decorative element can instead be the emanating surface or form a part of such surface.

The method includes periodically replenishing the supply with additional liquid fragrance so that the fragrance can be emanated continuously. Also, the method contemplates changing the fragrance to one with a different odor character by removing and replacing the wicking members after the initial supply of fragrance is diffused, and by thereafter introducing a supply of a different fragrance in the support member in contact with the wicking member.

The invention also relates to the use of the fragrance dispenser disclosed herein for providing a fragrance to a botanical product provided on the holding member, with a particular and preferred botanical product being potpourri.

### BRIEF DESCRIPTION OF THE DRAWINGS

Preferred features of the invention are explained in the following detailed description when reviewed in combination with the appended dragging figures, wherein:

FIG. 1 is a perspective view of a holding member for use in the fragrance dispenser of the invention;

FIG. 2 is a cross-sectional view of the dispenser, illustrating the various components therein;

FIG. 3 is a perspective view of a support for use in the fragrance dispenser of the invention;

FIG. 4 is a partial cross-sectional view of the fragrance dispenser of the invention illustrating the presence of potpourri;

FIG. 5 is another is a cross-sectional view of the dispenser, illustrating the presence of liquid fragrance in the support member;

FIG. 6 is a perspective view of another holding member for use in the fragrance dispenser of the invention; and

FIG. 7 is a top view of the holding member of FIG. 1 to illustrate the arrangement of the wicking members thereon.

### DETAILED DESCRIPTION OF THE INVENTION

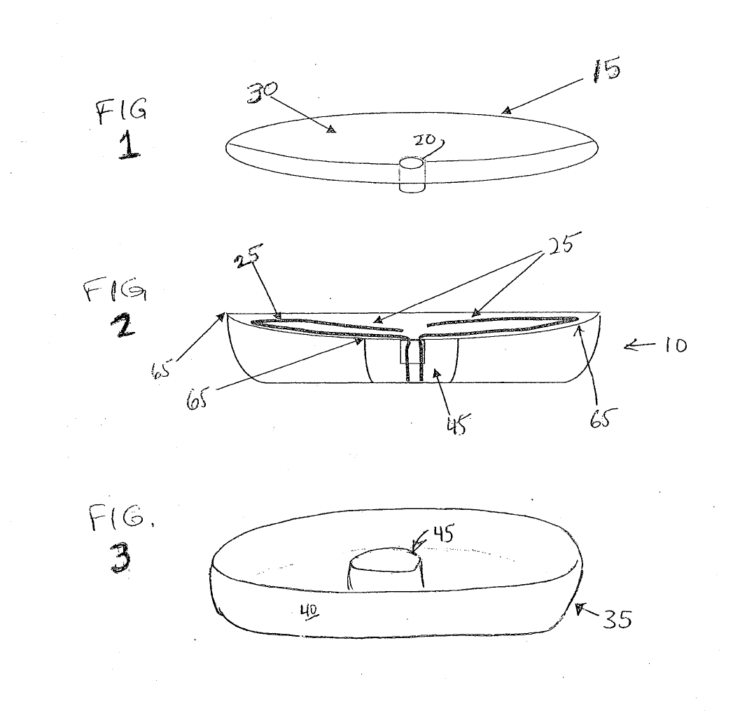

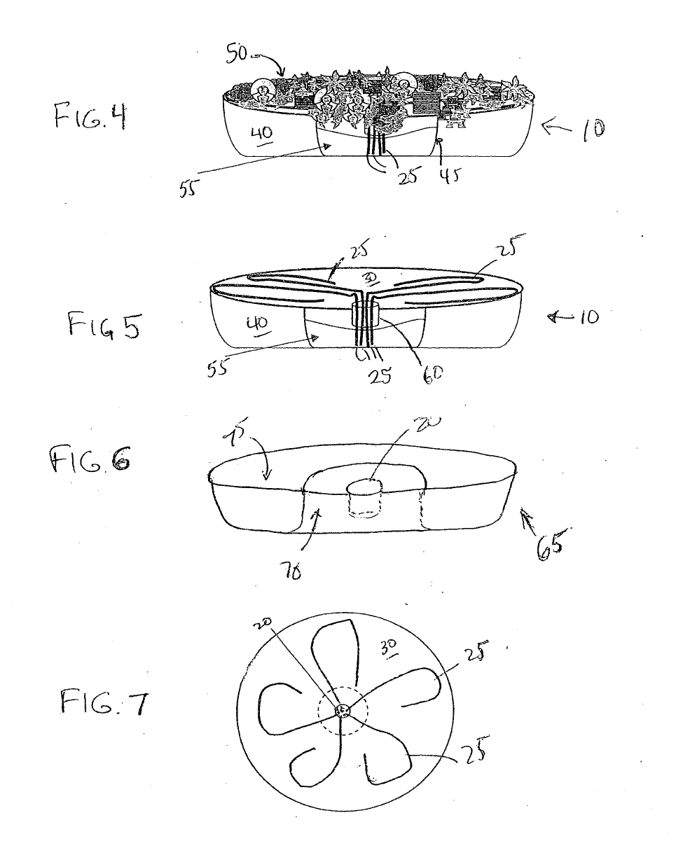

Referring initially to FIGS. 1-3, the fragrance dispenser of the invention 10 includes a potpourri holding member in the form of a plate 15 that preferably is shaped like a shallow platter with a central hole 20. The plate 15 serves a multiple purposes. It is used to hold the potpourri while also providing a surface 30 upon which wicking members 25 are supported or upon which the wicking members 25 can be arranged and placed for diffusion of the liquid fragrance. FIG. 3 shows a support member 35 in the form of a bowl shaped member having a cylindrical sidewall 40 and interior compartment 45 for holding a supply of liquid fragrance.

FIG. 4 illustrates the dispenser 10 with the addition of an aesthetically pleasing display of botanical product such as potpourri 50 thereon. The cross-sectional view of FIG. 4 shows the level of the liquid fragrance 55 in interior compartment 45, along with the extension of the wicking members 25 into the liquid fragrance.

The overall design of the product is intended to appeal to the taste of the consumer, such that the shape and final size of the product can vary. The preferred shape of these products is seen in the support member of FIG. 3, which is a round bowl or dish-like container having some degree of concavity. The most preferred size is approximately 10 inches in diameter with an overall depth of 1 to 4 inches.

The holding member 15 provides a surface 30 where the botanical products 50 sit without the risk of spillage, while the support member 35 in the form of a lower container or housing is designed to hold the liquid fragrance 55 in a compartment or reservoir 45 with a minimum risk of spillage, as shown in FIG. 5. The support member can take on various shapes and sizes, with its key features including that it can properly house the fragrance compartment without hindering fragrance diffusion and at the same time minimizing the risk of any spillage of the liquid fragrance. This housing also takes the shape of a bowl or dish and is approximately 10.25 to 12 inches in diameter, with a height of approximately 3 inches. The fragrance compartment 45 is centrally located within the bowl 35. The dimension of this compartment is approximately 3 inches diameter, with a height of approximately 2 inches.

The upper plate 15 also provides a surface 30 for supporting or being layered with a wicking device or substrate that will be in direct contact with the liquid fragrance as well as with the botanical product or potpourri. This substrate or wicking system will control the capillary mechanism of the fragrance coming from the refillable reservoir in the lower layer. This system is illustrated as a plurality of reed members that are configured and designed to have one end that extends into the liquid fragrance and an opposite end that is arranged upon the surface 30 of the top plate 15 to allow for diffusion of the fragrance. The absorbent wicking substrate can be made of paper, cotton, plastics such as cellulose acetate, wood, polyester, polyethylene, porous ceramics or any other materials that can absorb and release a liquid fragrance.

In a preferred embodiment the wicking substrate comprises a plurality of reed members. These members may be made of wood or rattan stick or of any wood material that is flexible and that has the ability to absorb and release fragrance in the desired manner. These reed sticks will fit onto the upper platter forming uniform layer around the platter, because of the flexibility of these sticks they can either be glued onto the platter and or preformed into a rigid design that can sit upon the surface. To avoid loss of the sticks when moving or transporting the dispenser, the reed sticks can be configured in such as manner as to form a hook-like mechanism which can snap below the periphery of the top plate 15. In this embodiment, one end of the sticks acts as an anchoring support to the plate while the other end will sit directly in the fragrance reservoir, wicking the liquid fragrance or oil onto the potpourri or other botanical ornaments on the top plate 15.

An additional feature in the design of these reed sticks to secure them to the dispenser is to provide the end that fits into fragrance reservoir with o-rings, valves or a sealant to maintain them in a set position throughout the life of the product. These o-rings, valves or sealants can be made from any suitable material, including plastic, rubber, polyethylene, cotton, paper, polycarbonate metals, ceramics, or even adhesives when the dispenser is designed for a single use. The preferred material is plastic or any suitable material that does react adversely with the liquid fragrance. The o-rings or valves are used rather than adhesives when the liquid fragrance is to be replenished or the reed sticks are to be replaced. A typical plastic valve 60 is illustrated in FIG. 5.

As mentioned the size and length of these rattan sticks can vary to fit the dimension of the holding and support members. A preferred length of these sticks can vary from 1-20 inches, the diameter of these stick varies between 0.05 to 5 inches, with the preferred dimension for each one being a length of 12 inches and a diameter of 0.2 inches.

The size and amount of absorbent in the wicking system will affect the rate of delivery hence the end of life of the product. As a result, the careful selection in the amount and the dimension of these substrate will correlate to rate of evaporation and directly to the fragrance diffusion. Using these rattan or reed sticks allows more flexibility in the shape and the quantity utilized. The amount of sticks will vary from 1-50, the preferred amount being 12 sticks of the previously recited size that which provide a delivery rate of approximately 2 to 5 grams per day depending on the volatility of the liquid fragrance or fragrance oil. If more fragrance release is desired, the size and/or amount of the sticks can be increased, as fragrance release is directly correlated to these variables.

The holding member 15 is designed with an aperture 20 and a connecting valve that fits therein so that the reed sticks 25 extend directly into the fragrance container 45 and liquid fragrance 55. The aperture or hole 20 will also serve as the central point to refill the fragrance oil into the fragrance container. This aperture or hole is also the insertion point for the wicking substrate into fragrance reservoir, which will be attached or fitted onto the fragrance reservoir.

The fragrance reservoir 45 can be made as part of the bowl 35 itself to hold liquid fragrance therein, or it can be designed as a housing for receiving and holding a separate fragrance reservoir. For the former design, to add further fragrance, all that is needed is to remove the reed sticks 25 and/or valve (if one is used) and then pour additional liquid fragrance through hole 20 and into compartment 45. The reed sticks and valve can then be replaced. Alternatively, when the fragrance reservoir 45 is to hold a separate bottle of liquid fragrance, the top plate 15 can be removed and the bottle replaced.

Referring again to FIGS. 1-3, the holding 15 and support 35 members can be structured to be removable, for the plate member 15 shown in FIG. 1, the preferred design is that the top and bottom bowls are attached as a single unit. This attachment is made at the periphery of the top plate 15 where it meets the top of the cylindrical sidewall 40 of the support member, as shown in FIG. 2 by numeral 65. Thus, the consumer only needs to refill the fragrance through the aperture 20 in the top plate 15.

The refillable aspect of this invention is an important feature and can take on several design features, such as the refillable bottle can be inserted from the bottom of lower bowl or holding container instead of through the top, as desired. For this embodiment, the top plate 15 and support bowl 35 can be sealed together as the fragrance bottle can be received in compartment 45 either loosely, or if a more secure connection is desired, by a press fit or even a screw type design. Of course, the bowl 35 can also be provided with a door or removable panel to allow access to compartment 45 and to retain the bottle therein after it is replaces a spent liquid fragrance bottle.

The bottom bowl 35 may be transparent to provide an attractive feature in the design to allow the customer to see the physical or visual emptying of the liquid fragrance or fragrance oil. This is optional, however, as one can view the fragrance reservoir from the aperture in the top bowl to determine when it needs to be replaced or replenished. The bowl member 35 as well as the top plate 15 can be made from glass, metals, plastics clay, stainless steel, aluminum, ceramics or any other suitable materials that will not react negatively with fragrance oils.

FIG. 6 illustrates an alternative design for the holding member instead of the top plate 15. In this embodiment, the holding member 65 is shaped more like the support bowl 35 and with an interior cover section 70 for surrounding and receiving the internal compartment 45 of the bowl 35. This allows the support member 35 to nestingly receive holding member 65 so that it is not necessary to seal the two together. Removal of the holding member 65 can be achieved to provide access to the internal compartment 45 of the support member 35 to allow for replenishment or replacement of the liquid fragrance.

Additional optional features that can be provided to improve consumer appeal. One such feature is a completely removable wicking substrate that can be placed onto the top plate 15 and into the liquid fragrance 35. Without anchoring the wicking substrate to the upper platter or bowl, it can be easily removed and disposed or replaced. This substrate can be a plurality of the reed sticks mentioned herein or a structure of any other absorbent material. Since the wicking substrate sits directly onto the holding member 15, the substrate does not need to support its own weight. Also, by being removable, the consumer can change the wicking substrate along with botanical product when necessary or as desired. The floral or botanical arrangement along with the fragrance can thus be changed to fit the decor or color of the room or space. This interactive feature allows the end users more choice at the point of purchase, i.e., the dispenser can be a do it yourself kit when initially purchased or it can be modified during use by replacement of the wicking members and fragrance to different odors or floral arrangements as desired.

Another feature is that the wicking substrate can be a smaller member connected directly into the fragrance bottle. So when fitted in this manner, the provision of the fragrance bottle into the device enables the short wicking members to protrude out of the hole 20 in the top plate 15. While this can be used alone for diffusing the fragrance, for optimal diffusion, the short wicking member can be arranged to contact another absorbing substrate on the surface 30 of the top plate 15 then as noted holds the botanical product or potpourri. This second absorbing substrate can then be removable and disposable to provide greater versatility to change to a different fragrance or to display new botanicals without any cross contamination of fragrances from the previously used one.

What is claimed is:

1. A fragrance dispenser comprising:
a holding member having a surface for holding a product, decorative element, ornament or emanating surface;
a support member for supporting the holding member and for receiving a supply of liquid fragrance; and
a plurality of wicking members extending from the supply of liquid fragrance to and upon the holding member surface for transferring the liquid fragrance from the supply to the surface for diffusing the fragrance therefrom.

2. The dispenser of claim 1, further comprising a product, decorative element, ornament or emanating surface on the holding member.

3. The dispenser of claim 1, wherein the supply of liquid fragrance is received in a fragrance chamber provided in the support member.

4. The dispenser of claim 3, wherein the holding member is bowl shaped to retain the product therein or thereon and the support member is bowl shaped to support the holding member.

5. The dispenser of claim 3, wherein the holding member is a dish or platter having a hole through which the wicking members pass to reach the supply of liquid fragrance.

6. The dispenser of claim 5, wherein the supply of liquid fragrance is provided in a bottle and the fragrance chamber is mounted centrally in the support member beneath the hole in the holding member.

7. The dispenser of claim 6, wherein the holding member has a generally circular perimeter and the plurality of wicking members comprise reed members having first ends configured to extend into the supply of liquid fragrance and having second ends configured conform to the holding member surface and extend at least halfway towards the perimeter of the holding member.

8. The dispenser of claim 7, wherein the second ends of the reed members extend to near the perimeter of the holding member and then in a different direction to extend over additional portions of the holding member surface for greater diffusion of the fragrance therefrom.

9. The dispenser of claim 7 wherein between 2 and 50 wicking members are provided each having a diameter of about 0.05 to 0.5 inches and have a length sufficient to diffuse the desired amount of liquid fragrance.

10. The dispenser of claim 1, wherein the wicking members are made of paper, a fabric or a polymer material capable of absorbing and releasing the fragrance and the holding member and support member are each made of plastic, ceramic, metal or other materials of combinations thereof such that the inner surface of the holding member does not absorb the liquid fragrance.

11. A method for enhancing product fragrance diffusion, which comprises:
providing the fragrance dispenser of claim 1;
adding a liquid fragrance to the support member; and
arranging the wicking members to contact the supply of liquid fragrance to transfer the liquid fragrance from the supply to the surface of the holding member for diffusing the fragrance therefrom.

12. The method of claim 11, which further comprises arranging a product, decorative element, ornament or emanating surface upon the holding member so that it appears that the product, decorative element, ornament or emanating surface is emanating the fragrance.

13. The method of claim 11, which further comprises periodically replenishing the supply with additional liquid fragrance.

14. The method of claim 11, which further comprises changing the fragrance to one with a different odor character by removing and replacing the wicking members after the supply of fragrance is diffused, and by introducing a supply of a different fragrance in the support member in contact with the wicking member.

15. Use of the fragrance dispenser of claim 1 for providing a fragrance to a product, decorative element, ornament or emanating surface provided on the holding member.
